(19) United States
(12) Patent Application Publication
(10) Pub. No.: US 2007/0021828 A1
(43) Pub. Date: Jan. 25, 2007

(54) Mechanically actuated stents and apparatus and methods for delivering them

(76) Inventors: Jeff Krolik, Campbell, CA (US); Elliot Kim, Santa Clara, CA (US)

Correspondence Address: Vista IP Law Group LLP, 2040 MAIN STREET, 9TH FLOOR, IRVINE, CA, 92614, US

(21) Appl. No.: 11/439,717

(22) Filed: May 23, 2006

Related U.S. Application Data
(60) Provisional application No. 60/683,931, filed on May 23, 2005.

(51) Int. Cl. A61F 2/06

(52) U.S. Cl. 623/1.31

(57) ABSTRACT

A stent is provided for deployment into an ostium communicating from a main vessel to a branch vessel. The stent includes a first tubular portion advanceable into the ostium that is expandable from a contracted condition to an expanded condition for dilating a lesion within the ostium. The stent includes a second tubular portion that may be flared radially outwardly to contact the ostium. The stent may be carried on a delivery apparatus including an actuator for expanding the second tubular portion, and one or more balloons for expanding the first distal portion and/or further expanding the proximal portion.

This application claims benefit of provisional application Ser. No. 60/683,931, filed May 23, 2005, the entire disclosure of which is expressly incorporated by reference herein.

### FIELD OF THE INVENTION

The present invention relates generally to endoluminal prostheses or “stents,” and, more particularly, to mechanically actuated flared stents, and to apparatus and methods for delivering such stents into an ostium of a blood vessel or other body lumen.

### BACKGROUND

Tubular endoprosthesis or “stents” have been suggested for dilating or otherwise treating stenoses, occlusions, and/or other lesions within a patient's vasculature or other body lumens. For example, a self-expanding stent may be maintained on a catheter in a contracted condition, e.g., by an overlying sheath or other constraint, and delivered into a target location, e.g., a stenosis within a blood vessel or other body lumen. When the stent is positioned at the target location, the constraint may be removed, whereupon the stent may automatically expand to dilate or otherwise line the vessel at the target location. Alternatively, a balloon-expandable stent may be carried on a catheter, e.g., crimped or otherwise secured over a balloon, in a contracted condition. When the stent is positioned at the target location, the balloon may be inflated to expand the stent and dilate the vessel.

Sometimes, a stenosis or other lesion may occur at an ostium or bifurcation, i.e., where a branch vessel extends from a main vessel or trunk. For example, such a lesion may form within a coronary artery immediately adjacent the aortic root. U.S. Pat. No. 5,749,890 to Shaknovich discloses a stent delivery assembly for placing a stent in an ostial lesion. U.S. Pat. No. 5,632,762 to Myler discloses a tapered balloon on a catheter for positioning a stent within an ostium. U.S. Pat. No. 5,607,444 to Lam discloses an expandable ostial stent including a tubular body and a deformable flaring portion. Published application US 2002/0077691 to Nachtigall discloses a delivery system that includes a sheath for holding a stent in a compressed state during delivery and a retainer that holds a deployable stop in an undeployed position while the delivery system is advanced to a desired location.

Accordingly, stents and apparatus and methods for delivering stents within an ostium would be useful.

### SUMMARY OF THE INVENTION

The present invention is directed to endoluminal prostheses or “stents,” and, more particularly, to mechanically actuated, flared stents, and to apparatus and methods for delivering such stents into an ostium of a blood vessel or other body lumen.

In accordance with one embodiment, a stent is provided that includes a first tubular portion and a second flaring portion. The first portion may include a length, and may be expandable from a contracted condition to an expanded condition. The second portion may include a first annular band disposed adjacent the first tubular portion and a second annular band disposed adjacent the first tubular portion. The second tubular portion may be configured such that, upon application of an axial compressive force, the first and second annular bands buckle outwardly at a location between the first and second annular bands. In one embodiment, the second tubular portion may be further configured such that the second annular band expands into a ring upon application of a radially outward expansion force.

In accordance with another embodiment, a stent is provided that includes a first tubular portion including a length, the first tubular portion being expandable from a contracted condition to an expanded condition, and a second tubular portion. The second portion may include a first annular band disposed adjacent the first tubular portion and a second annular band disposed adjacent the first tubular portion. The second annular band may include a plurality of axial elements connected by alternating curved elements, the second tubular portion being configured such that, upon application of an axial compressive force, the first and second annular bands buckle outwardly at a location between the first and second annular bands. In one embodiment, the second annular band may be configured such that, upon application of a radially outward expansion force, the curved elements at least partially straighten such that the axial elements at least partially define a circle or ellipse.

In accordance with still another embodiment, an apparatus is provided for treating an ostium communicating between a main body lumen and a branch body lumen. Generally, the apparatus includes an elongate member including proximal and distal ends, an expandable member on the distal end that is expandable from a collapsed configuration to an expanded configuration, a stent on the distal end, and an actuator movable relative to the distal end for buckling a first flaring portion of the stent when the actuator is activated. In one embodiment, the first flaring portion may include first and second annular bands, the first flaring portion configured to buckle radially outwardly between the first and second annular bands when the actuator is activated.

Other aspects and features of the present invention will become apparent from consideration of the following description taken in conjunction with the accompanying drawings.

### BRIEF DESCRIPTION OF THE DRAWINGS

The drawings illustrate exemplary embodiments of the invention, in which:

FIG. 1 is a perspective view of an exemplary embodiment of a mechanically actuated stent in an expanded, flared condition.

FIG. 2 is a top view of a portion of a cell pattern for a mechanically actuated flared stent that may be expanded into an enlarged, flared condition, such as that shown in FIG. 1.

FIGS. 3A-3C are side views of an exemplary embodiment of a delivery catheter carrying a stent, showing one end of the stent being buckled to expand from a contracted condition to an enlarged, flared condition.

FIGS. 3D-3H are perspective views of the stent of FIGS. 3A-3C being further expanded within a body lumen communicating with an ostium.

FIGS. 4A-4C are schematic side views of another embodiment of a delivery catheter carrying a stent, showing one end of the stent being buckled to expand from a contracted condition to an enlarged, flared condition.

FIG. 5 is a detail, showing a mechanism for mechanically actuated a stent to cause cells of the stent to expand and buckle.

FIGS. 6A and 6B are schematic side views of yet another embodiment of a delivery catheter carrying a stent, showing one end of the stent being buckled to expand from a contracted condition to an enlarged, flared condition.

FIG. 7 is a detail, showing an alternative mechanism for mechanically actuating a stent.

FIGS. 8A and 8B are schematic side views of yet another embodiment of a delivery catheter carrying a stent, showing one end of the stent being buckled to expand from a contracted condition to an enlarged, flared condition.

FIGS. 8C-8E are schematic side views of the delivery catheter of FIGS. 8A and 8B, showing the stent being radially expanded.

FIGS. 9A and 9B are schematic side views of still another embodiment of a delivery catheter carrying a stent, showing the stent being buckled and expanded from a contracted condition to an enlarged, flared condition.

FIG. 10 is a detail of a mechanism that may be provided on a delivery catheter for capturing a portion of a stent to allow the stent to be mechanically actuated.

FIGS. 11A and 11B are details, showing the mechanism of FIG. 10 engaging portions of a stent to allow the stent to be mechanically actuated.

FIG. 12 is a detail of another mechanism that may be provided on a delivery catheter for capturing a portion of a stent to allow the stent to be mechanically actuated.

FIG. 13 is a detail, showing the mechanism of FIG. 12 engaging a portion of a stent to allow the stent to be mechanically actuated.

FIGS. 14A-14D are side views of a stent carried on a delivery catheter, showing a method for expanding the stent from a contracted condition to an expanded, flared condition.

FIG. 15 is a cross-sectional view of an ostium communicating between a main vessel and a branch vessel.

FIG. 16 is a graph showing desired properties of a stent relative to the ostium shown in FIG. 15.

FIGS. 17-20 are top views of various cell patterns that may be provided for a stent including a flaring portion and having variable properties along its length.

FIGS. 20A-20C are details showing various connectors that may be provided on a stent for connecting adjacent bands of cells.

FIG. 21 is a top view of another cell pattern that may be provided for a stent including a flaring portion and having variable properties along its length.

FIGS. 22A-22F are side views of a distal end of a delivery catheter, showing a method for expanding a flaring stent.

### DETAILED DESCRIPTION OF THE PREFERRED EMBODIMENTS

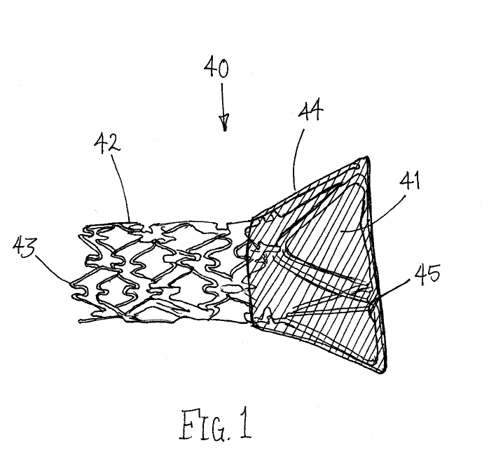

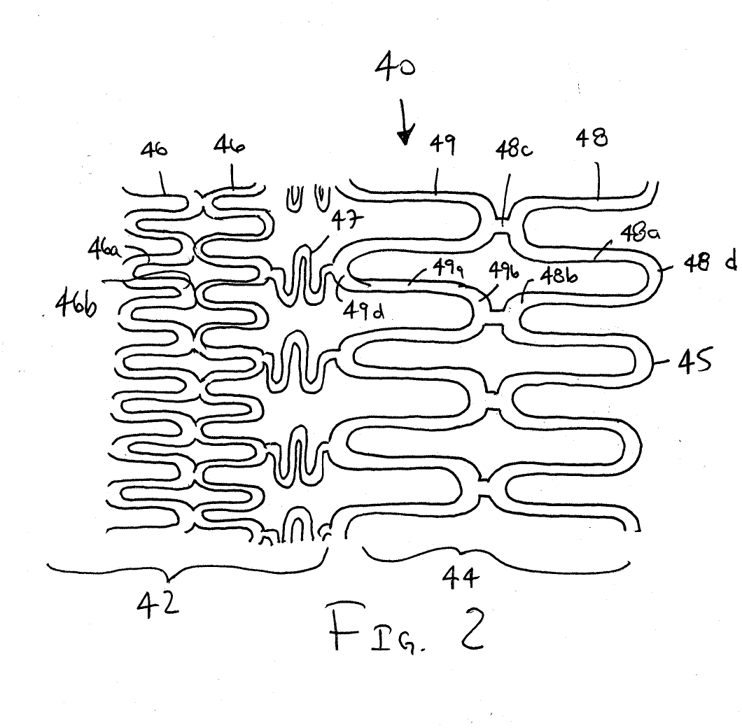

Turning to the drawings, FIG. 1 shows an exemplary embodiment of a stent apparatus 40 that includes a generally cylindrical distal or first portion 42 and a flared proximal or second portion 44. With additional reference to FIG. 2, the stent 40 may include a plurality of annular bands 46-49 connected to adjacent bands between first and second ends 43, 45 of the stent. In addition or alternatively, the stent 40 may include a plurality of cells that may be connected to one another around a circumference and/or along a length of the stent 40.

For example, as shown in FIG. 2, the first portion 42 of the stent 40 may include a plurality of annular bands (two exemplary bands 46 being shown) defined by zigzag or serpentine patterns of straight elements 46a whose ends are connected alternately by curved elements 46b extending about the circumference of the stent 40. The zigzag pattern of the bands 46 may include straight elements 46a having similar lengths (providing a predetermined amplitude or length for each band 46) and/or may include similar numbers of curved elements 46b around the circumference (providing a predetermined period around the circumference). As shown, the first portion 42 of the stent 40 has a substantially homogenous cell structure. Alternatively, other, non-uniform cell and/or band configurations may be provided, if desired. Any number of annular bands 46 may be provided, e.g., to provide a first portion 42 having a desired length, e.g., corresponding to a length of a lesion being dilated or otherwise treated using the stent 40.

A band of transition elements 47 may connect the first and second portions 42, 44 of the stent 40. The transition elements 47 may include one or more sinusoidal or other curved segments that extend generally axially, as shown. Alternatively, the transition elements 47 may be substantially straight axial segments (not shown), depending upon the desired flexibility between the first and second portions 42, 44.

The second portion 44 of the stent 40 may include a first annular band 48 immediately adjacent the second end 45, including a zigzag or serpentine pattern defined by a plurality of straight elements 48a whose ends are connected alternately by curved elements 48b, 48d extending around the circumference of the stent 40. The straight elements 48a of the first annular band 48 may have longer lengths (amplitudes) than the straight elements 46a and/or the zigzag pattern may include fewer curved elements 48b (i.e., may have a longer period) than the curved elements 46b included in the annular bands 46 of the first portion 42.

The second portion 44 may also include a second annular band 49 adjacent the first annular band 48, which may have similar amplitudes and/or periods than the first annular band 48, e.g., including similar straight elements 49a and/or alternating curved elements 49b, 49d. As shown, the second annular band 49 is offset one hundred eighty degrees (180°) from the first annular band 48 such that pairs of curved elements 48b, 49b are disposed axially adjacent one another.

A link 48c may be provided that connects axially adjacent curved elements 48b, 49b of the first and second annular bands 48, 49. The link 48c may have a width and/or thickness that is smaller than the elements (e.g., the straight elements 48a, 49a and/or curved elements 48b, 49b) of the first and second annular bands 48, 49. The links 48c may preferentially buckle when the first and second annular bands 48, 49 are subjected to an axially compressive force, as described further below.

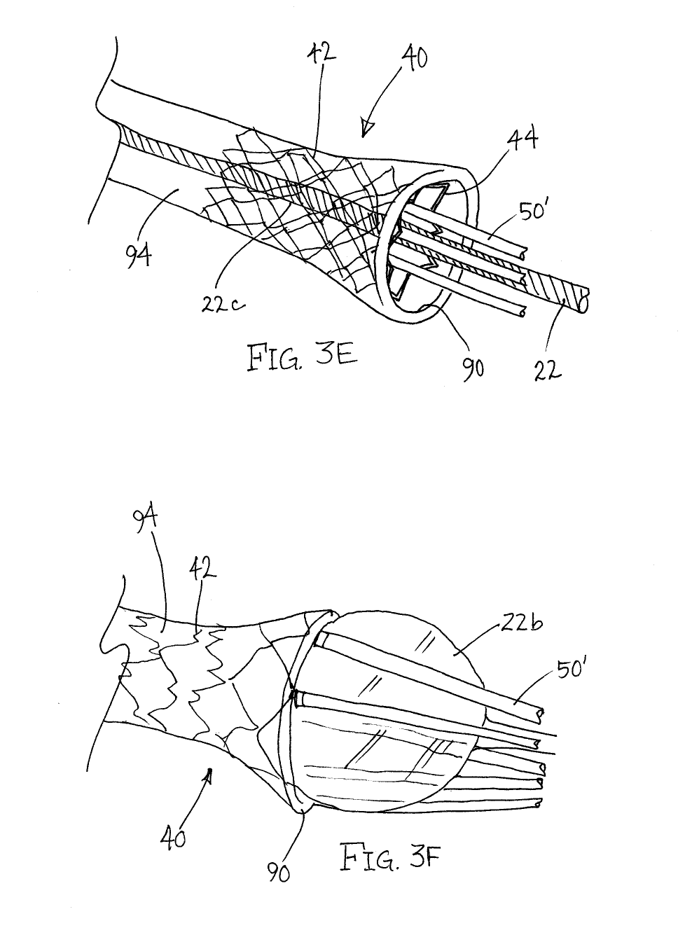

The stent 40 may be formed from a variety of materials that may be plastically deformed to allow expansion of the stent 40. For example, the stent 40 may be formed from metal, such as stainless steel, tantalum, MP35N, Niobium, Nitinol, and L605, plastic, or composite materials. In particular, the materials of the stent 40 may be plastically deformed under the pressures experienced when the stent 40 is expanded, e.g., such that the first and/or second portions 42, 44 of the stent 40 are deformed beyond their elastic limit. Thus, when the stent 40 is deployed, the stent 40 may maintain its expanded configuration (e.g., that shown in FIG. 4C) with minimal recoil. Stated differently, the stent 40 material may resist collapsing back towards its reduced configuration after deployment, e.g., if the tissue surrounding the body lumen attempts to constrict or otherwise return to its occluded shape.

Alternatively, at least a portion of the stent 40 may be self-expanding. For example, one or both of the first and second portions 42, 44 may be biased to expand at least partially outwardly yet may be constrained on a delivery device in a contracted condition to facilitate delivery. In this alternative, the stent 40 may be formed from Nitinol or other shape memory or superelastic materials.

Optionally, the resistance of the stent 40 to expansion may be varied along its length. This performance of the stent 40 may be based upon mechanical properties of the material, e.g., which may involve heat treating one or more portions of the stent 40 differently than other portions. In addition or alternatively, the structure of the stent 40 may be varied, e.g., by providing struts, fibers, or other components in different portions having different widths, thicknesses, geometry, and the like. In one embodiment, the material of the first portion 42 may require greater or less force to expand than the second portion 44.

Additional information on methods for making and/or using the stent 40, and/or alternative configurations for the first portion 42 or other components of the stent 40 may be found in co-pending applications Ser. Nos. 60/710,521, filed Aug. 22, 2005, 60/731,568, filed Oct. 28, 2005, 60/757,600, filed Jan. 9, 2006, 60/743,880, filed Mar. 28, 2006, and 60/745,177, filed Apr. 19, 2006. The entire disclosures of these references are expressly incorporated by reference herein.

The stent 40 may be a generally tubular structure, e.g., including openings in a tubular wall that facilitate expansion of the stent 40 and/or allow tissue ingrowth. For example, the stent may be an elongate tube that has slots or other openings formed in the tube wall, e.g., by laser cutting, mechanical cutting, chemical etching, machining, and the like. Alternatively, the stent 40 may be a braided or other structure, e.g., formed from one or wires or other filaments braided or otherwise wound in a desired manner. Additional possible stent structures may include helical coil wires or sheets.

If desired, one or more portions of the stent 40 may include a membrane, film, or coating (not shown), e.g., to create a nonporous, partially porous, or porous surface between cells of the stent 40. For example, as shown in FIG. 1 the second portion 44 of the stent 40 may include a substantially elastic membrane 41, e.g., PTFE, ePTFE, silicone, polyurethane, or polyethylene, that may be embedded into, coated onto, sandwiched around, or otherwise carried by the stent 40. The membrane 41 may be substantially elastic such that the membrane 41 may expand when the second portion 44 is flared or otherwise expanded. Alternatively, the membrane 41 may be folded or otherwise compressed such that the membrane 41 may unfold or otherwise accommodate expansion as the stent 40 is expanded.

The membrane 41 may be provided on an outer and/or inner surface of the second portion 44. A membrane 41 on the inner surface may facilitate recrossing the stent 40 at a later time after implantation. For example, after the stent 40 is implanted within a patient, it may be desirable to advance a guidewire or other instrument (not shown) through the ostium into the branch vessel, e.g., to perform another procedure. This may occur during the same surgical procedure, or some time after the patient has recovered, e.g., when the branch vessel, lesion, or main vessel need subsequent treatment. The membrane 41 may prevent the tip of a guidewire or other instrument from catching or tangling in the struts, cells, wires, or other structures of the stent 40. Instead, the membrane 41 may provide a substantially smooth, possibly lubricious surface that may guide a guidewire through the stent 40 into the branch vessel.

In addition or alternatively, a membrane 41 on the stent 40 may carry therapeutic or other compounds or materials. For example, a membrane 41 on an outer surface of the stent 40 may be pressed into contact with the plaque, damaged tissue, or other material of the lesion, allowing the compound to act to enhance healing or otherwise treat the lesion.

Optionally, the stent 40 may include one or more radiopaque or other markers (not shown), e.g., to facilitate monitoring the stent 40 during advancement, positioning, and/or expansion. For example, a band of radiopaque material, e.g., gold, platinum, iridium, tungsten, or their alloys, may be provided on each end of the stent 40 and/or adjacent the location where the first and second portions 42, 44 meet. In addition or alternatively, wires, rods, disks, or other components (not shown) may be provided on predetermined locations on the stent 40 that are formed from radiopaque material to facilitate monitoring the stent 40 using fluoroscopy or other external imaging.

In addition or alternatively, the stent 40 may carry one or more therapeutic or other compounds (not shown) that may enhance or otherwise facilitate treatment of a target location within a patient's body. For example, the stent 40 may carry compounds that prevent restenosis at the target location.

Turning to FIGS. 4A and 4B, an exemplary embodiment of an apparatus 10 is shown for delivering the stent 40 (which may be any of the embodiments described herein) to a desired location, e.g., within an ostium and/or branch vessel (not shown). Generally, the apparatus 10 includes a delivery catheter 12 and a pusher or other actuator 50 for expanding or otherwise deploying the stent 40, as described further below. The delivery catheter 12 generally includes a proximal end 14, a distal end 16, and one or more lumens extending between the proximal and distal ends 14, 16, thereby defining a longitudinal axis 20 between the proximal and distal ends 14, 16. The delivery catheter 12 includes one or more balloons or other expandable members 22 on the distal end 16 of the delivery catheter 12 for expanding and/or deploying the stent 40, as described further below. Optionally, the delivery catheter 12 may include a locator device (not shown) on the distal end 16, e.g., proximal or otherwise adjacent to the stent 40. Exemplary locator devices and methods for using them are disclosed in co-pending application Ser. No. 60/683,931, filed May 23, 2005, entitled “Apparatus and Methods for Locating an Ostium of a Vessel,” the entire disclosure of which is expressly incorporated by reference herein.

The delivery catheter 12 may be formed from one or more tubular bodies, e.g., having variable flexibility along its length. For example, the distal end 16 may be substantially flexible to facilitate insertion through tortuous anatomy, e.g., terminating in a rounded, tapered, and/or other substantially atraumatic distal tip 17. The distal end 16 may be sized and/or shaped for introduction into a body lumen, e.g., having a diameter between about one and seven millimeters (1-7 mm), or less than 1.5 millimeters. The proximal end 14 may be substantially flexible or semi-rigid, e.g., having sufficient column strength to facilitate advancing the distal end 16 through a patient's vasculature by pushing on the proximal end 14. The delivery catheter 12 may be formed from plastic, metal, or composite materials, e.g., a plastic material having a wire, braid, or coil core, which may preventing kinking or buckling of the catheter 12 during advancement.

The delivery catheter 12 may include a handle 30 on the proximal end 14, e.g., to facilitate manipulating the delivery catheter 12. The handle 30 may include one or more side ports 32 communicating with respective lumens within the delivery catheter 12, e.g., a side port 32b communicating with a lumen (not shown) communicating with an interior of the balloon 22. The handle 30 may be molded, machined, or otherwise formed from plastic, metal, or composite material, e.g., providing an outer casing, which may be contoured or otherwise shaped to ease manipulation. The proximal end 14 of the delivery catheter 12 may be attached to the handle 30, e.g., by bonding, cooperating connectors, interference fit, and the like. Optionally, if the apparatus includes any actuatable components (not shown) on the distal end 16, the handle 30 may include one or more actuators (not shown), such as one or more slides, dials, buttons, and the like, for actuating or otherwise manipulating the components on the distal end 16 from the proximal end 14, as explained further below.

In the embodiment shown in FIGS. 4A-4C, the delivery catheter 12 includes at least two lumens extending between the proximal ends 14, 16. For example, the delivery catheter 12 may include a guidewire or instrument lumen (not shown) that extends from a side port 32a in the handle 30 to an opening 34 in the distal tip 17. The instrument lumen may have sufficient size to allow a guidewire or other rail or instrument (not shown) to be inserted therethrough, e.g., to facilitate advancing the delivery catheter 12 over the rail, as explained further below. Optionally, the handle 30 may include one or more seals (not shown) within or adjacent the port 32a, e.g., a hemostatic seal that prevents fluid, e.g., blood, from flowing proximally out of the port 32a, yet allows one or more instruments to be inserted therethrough and into the instrument lumen.

In addition, the delivery catheter 12 may include one or more inflation lumens that extend from respective side port(s) 32b in the handle 30 through the delivery catheter 12 to openings (not shown) that communicate with an interior of a respective balloon 22. The side port(s) 32b on the handle 30 may include connectors, e.g., a luer lock connector (not shown), one or more seals (also not shown), and the like. A source of inflation media and/or vacuum, e.g., a syringe filled with saline (not shown), may be connected to the side port(s) 32b, e.g., via tubing (also not shown), for expanding and/or collapsing the balloon 22.

As shown in FIGS. 4A-4C, the delivery catheter 12 includes one balloon 22 on the distal end 16. Alternatively, the delivery catheter 12 may include multiple balloons (not shown) on the distal end 16 over which the stent 40 may be placed. Additional information on multiple balloon catheters and methods for using them are disclosed in co-pending application Ser. No. 11/136,266, filed May 23, 2005, the entire disclosure of which is expressly incorporated by reference herein.

The balloon (or balloons, not shown) 22 may be bonded or otherwise secured to the distal end 16 of the delivery catheter 12. For example, ends of the balloon 22 may be attached to the distal end 16 using one or more of bonding with an adhesive, sonic welding, an annular collar or sleeve, and the like. The balloon 22 may be expandable from a contracted condition (not shown), which may facilitate advancement through a patient's vasculature to an enlarged condition for expanding or otherwise deploying the stent 40.

The balloon(s) 22 may be formed from substantially inelastic material, e.g., PET, nylon, or PEBAX, such that the balloon 22 expands to a predetermined size in its enlarged condition once sufficient fluid is introduced into the interior of the balloon 22. Alternatively, the balloon 22 may be formed from substantially elastic material, e.g., silicone, polyurethane, or polyethylene, such that the balloon 22 may be expanded to a variety of sizes depending upon the volume and/or pressure of fluid within the interior.

With continued reference to FIGS. 4A-4C, the pusher 50 may include an elongate member slidably coupled to the delivery catheter 12. For example, as shown, the pusher 50 may include an elongate tubular member disposed around the delivery catheter 12. The pusher 50 may include a proximal end 52 disposed adjacent to and/or coupled to the handle 30 on the delivery catheter 12, and a distal end 54 disposed adjacent to the balloon 22 and/or stent 40 on the distal end 16 of the delivery catheter 12.

From the proximal end 52, the pusher 50 may be directed distally relative to the delivery catheter 12, as shown in FIGS. 4B and 4C, such that the distal end 54 abuts and/or otherwise engages the proximal portion 44 of the stent 40. Alternatively, the pusher 50 may be directed distally using a slider or other actuator (not shown) on the handle 30 that may be coupled to the pusher 50, e.g., by a wire, cable, or other mechanism (not shown).

One or more elements (not shown) may be provided on the distal end 16 of the delivery catheter 12 for securing or otherwise preventing a portion of the stent 40 from moving distally on the distal end 16. For example, as explained further below, stops, detents, hooks, or other elements (not shown) may be provided that engage the stent 40, e.g., at the second annular band 49, transition band 47 (see FIG. 2), or elsewhere on the stent 40 to prevent the stent 40 from moving distally, e.g., off of the balloon 22.

During use, as shown in FIGS. 4B and 4C the pusher 50 may be directed distally against the stent 40, thereby subjecting the stent 40 to a compressive axial force. This force causes the proximal portion 44 of the stent to at least partially buckle outwardly, as explained further below. As shown, the distal end 54 of the pusher 50 includes a collar or sleeve that abuts the proximal end 45 of the stent 40 when the pusher 50 is advanced. Optionally, the pusher distal end 54 may include one or more features, e.g., clasps, detents, hooks, and the like (not shown), that interlock or otherwise releasably connect to the stent 40, e.g., to the proximal end 45 of the stent 40, similar to other embodiments described elsewhere herein. The features may disengage from the stent 40 simply by pulling the pusher 50 proximally, e.g., after expanding the proximal portion 44 of the stent 40.

Alternatively, the features may be releasable upon activating an actuator on the proximal end 52 of the pusher 50 and/or on the handle 30, e.g., independent of axial movement of the pusher 50. This alternative may allow the proximal portion 44 of the stent 40 to be collapsed back to the contracted condition, if desired, e.g., to remove and/or discontinue delivery of the stent 40. For example, if a user expands the proximal portion 44 within a trunk, but then decides not to deliver the stent 40, the pusher member 50 may be pulled proximally, thereby collapsing the proximal portion 44 back to the contracted condition. The stent 40 may then be removed or directed to another location for expansion and delivery. In this alternative, the delivery catheter 12 may include one or more features, e.g., hooks, detents, stops, and the like (not shown), that prevent proximal movement of the distal end 43 of the stent 40 when the pusher 50 is pulled proximally, thereby subjecting the stent 40 to an axial tensile force that may allow plastic deformation of the proximal portion 44 of the stent 40 back to the contracted condition.

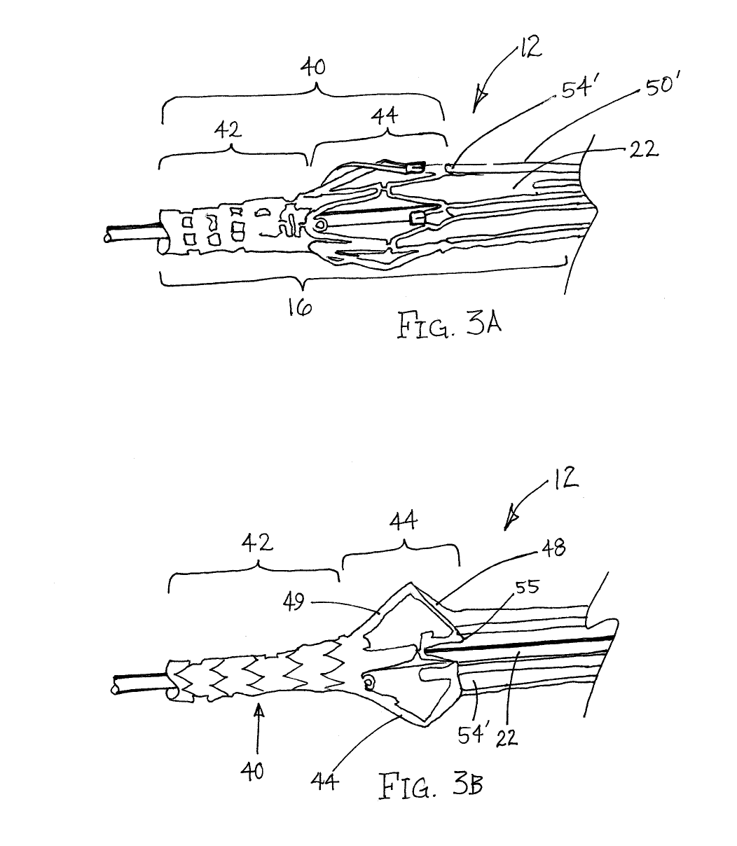

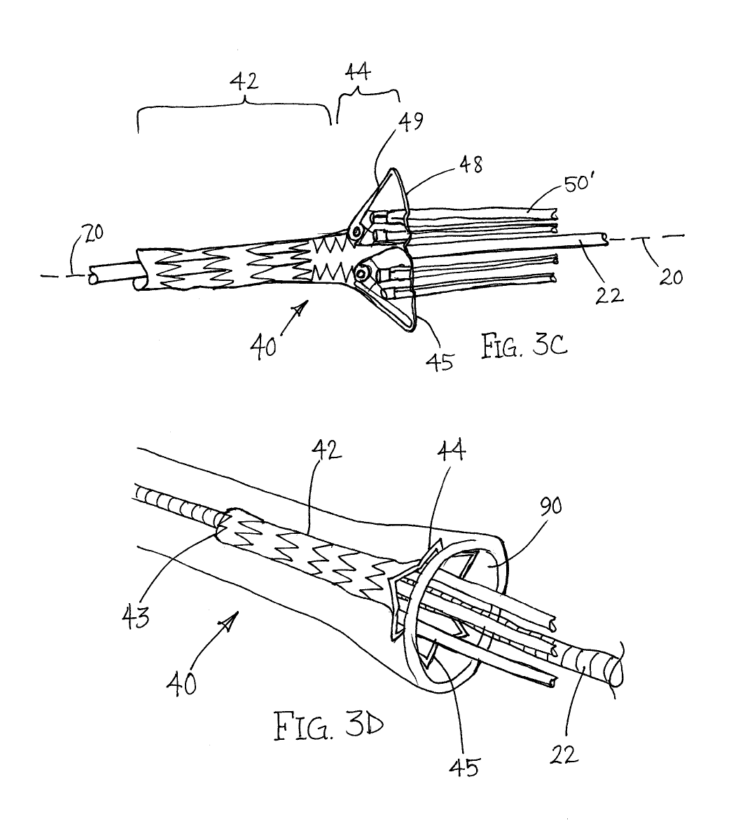

Turning to FIGS. 3A-3H, a method for delivering a stent 40, such as that shown in FIGS. 1 and 2, into an ostium 90 is now described. The ostium 90, a model of which is shown in FIGS. 3D-3H, may be an opening in a wall of a first or main body lumen or trunk (not shown) that communicates with a second body lumen or branch 94. In an exemplary embodiment, the trunk may be the aortic root and the branch 94 may be a coronary artery. In another embodiment, the trunk may be the distal aorta, and the branch 94 may a renal artery or other abdominal branch. It will be appreciated that the apparatus and methods described herein may be applicable to a variety of bifurcations or branches that extend transversely, e.g., laterally (at relatively shallow angles) or substantially perpendicularly, from another body lumen or trunk, e.g., within a patient's vasculature or other systems.

An occlusion or other lesion (not shown) may exist at and/or adjacent to the ostium 90, e.g., extending at least partially into the branch 94. The lesion may include atherosclerotic plaque or other material that partially or completely obstructs blood or other fluid flow between the trunk and the branch 94.

Initially, a guidewire or other rail (not shown) may be introduced from the trunk and through the ostium 90 into the branch 94 using conventional methods. For example, a percutaneous puncture or cut-down may be created at a peripheral location (not shown), such as a femoral artery, carotid artery, or other entry site, and the guidewire may be advanced through the patient's vasculature from the entry site, e.g., alone or with the aid of a guide catheter (not shown). Optionally, after the guidewire is directed into the branch 94 beyond the lesion, it may be desirable to at least partially dilate or otherwise treat the lesion. For example, an angioplasty catheter (not shown) may be advanced through the guide catheter and/or over the guidewire into and through the lesion, whereupon a balloon or other element on the catheter may be expanded to at least partially dilate the lesion. If desired, other procedures may also be performed at the lesion, e.g., to soften, remove, or otherwise treat plaque or other material forming the lesion, before the stent 40 is implanted. After completing any such procedures, instruments advanced over the guidewire may be removed.

If a guide catheter is used, the distal end of the guide catheter may be advanced over the guidewire into the trunk, e.g., until the distal end is disposed adjacent or proximal to the ostium 90. A distal end 16 of the delivery catheter 12 may be advanced over the guidewire and/or through the guide catheter from the entry site into the trunk. Optionally, the guide catheter may be partially retracted to expose the balloon 22 and stent 40, e.g., as shown in FIG. 3A.

Turning to FIG. 3B, the actuator 50′ may be activated from the proximal end (not shown) of the delivery catheter 12 to buckle and expand the proximal portion 44 of the stent 40. In the embodiment shown, the actuator 50′ includes a plurality of arms 54′ that engage or otherwise contact the proximal end 55 of the stent 40. The arms 54′ may be directed distally, while the stent 40 is maintained from moving distally, such that the proximal portion 44 buckles. For example, with additional reference to FIG. 2, the first and second annular bands 48, 49 may buckle outwardly causing links 48c to bend as the curved elements 48b, 49b move radially outwardly. Alternatively, other actuators and/or pusher members (not shown), such as those described elsewhere herein, may be used instead of the actuator 50.′

Turning to FIG. 3C, once the actuator 50′ is fully activated, the proximal portion 44 of the stent 40 may be flared outwardly, e.g., such that the second annular band 49 is flared or inclined, e.g., to define an obtuse angle with the longitudinal axis of the delivery catheter 12. The first annular band 48 may be oriented substantially perpendicularly or otherwise transversely relative to the longitudinal axis. In particular, because the curved elements 48d on the proximal end 45 of the stent 40 are engaged by the actuator arms 54,′ the curved elements 48d may remain adjacent the surface of the delivery catheter 12, while the curved elements 48b coupled to the links 48c are disposed outwardly away from the surface of the delivery catheter 12.

Turning to FIG. 3D, the distal end 16 of the delivery catheter 12 may then be advanced into the ostium 90 and/or the branch 94 from the trunk. If desired, a locator device (not shown) may be used to facilitate positioning the stent 40, as described in application Ser. No. 11/136,266, incorporated by reference above. Alternatively, the flared condition of the proximal portion 44 shown in FIG. 3C may provide a locator for positioning the stent 40 relative to the ostium 90. For example, the diameter of the proximal portion 44 in the flared condition may be selected to correspond to a size of the ostium 90, e.g., to be larger than the ostium 90, thereby allowing the stent 40 to be directed partially into the ostium 90 without passing entirely into the branch 94.

Turning to FIGS. 3E and 3F, the stent 40 may be further expanded within the ostium 90 and/or branch 94, e.g., to dilate or otherwise treat a lesion therein. For example, in the embodiment shown in FIG. 3E, the delivery catheter 12 includes a distal balloon 22a that may be inflated to expand the distal portion 42 of the stent 40. The distal balloon 22a may expand the distal portion 42 into a substantially uniform cylindrical shape or into a tapered shape, depending upon the shape of the distal balloon 22a selected and the anatomy encountered. As shown in FIG. 3F, a proximal balloon 22b may then be inflated to further expand the proximal portion 44 of the stent 40. In particular, this action may expand the first annular band 48 of the proximal portion 44, e.g., directing the curved elements 48d on the proximal end 45 of the stent 40 radially outwardly.

As best seen in FIG. 3H, this expansion may caused the curved elements 48d to at least partially straighten, e.g., as the straight elements 48a deform into a circumferential configuration, e.g., approximating a circle or ellipse extending around the ostium 90. Thereafter, as shown in FIG. 3G, the balloon(s) 22 may be deflated and the distal end 16 of the delivery catheter 12 withdrawn from the branch 94 and ostium 90, leaving the stent 90 in place. Optionally, the arms 54′ of the actuator 50′ may still engage the now-straightened curved elements 48d, thereby preventing the stent 40 from being dislodged while the delivery catheter 12 is withdrawn. The arms 54′ may be disengaged by directing the actuator 50′ proximally and/or by activating a release mechanism (not shown) on the handle 30 (also not shown) of the delivery catheter 12. As shown in FIG. 3H, the delivery catheter 12 and actuator 50′ may be removed from the patient, leaving the stent 40 within the ostium 90 and/or branch 94.

The resulting deployed condition of the stent 40 shown in FIG. 3H may provide a structure that is substantially resistant to the heavy elastic recoil expected when deploying in a large artery, such as the aorta or other parent vessel. The strength of the stent 40 is enhanced by the uninterrupted circle of struts 48a obtained when the first row of struts 48 are completely expanded during inflation of the proximal balloon 22b. Unlike conventional stents where a significant angle is maintained between adjoining struts to allow the balloon inflation, this stent 40 leaves a row of struts 48a aligned with each other end-to-end. In addition, the resulting structure may facilitate re-crossing the stent 40, e.g., should the patient ever need this ostium 90 to be accessed again by guidewire. Conventional stents may have numerous struts and flexible connections throughout their construction, which may present obstacles to attempts made to re-access the ostium 90 using a guidewire. In contrast, the stent 40 has relatively few struts 48a, 49a and flexible connections in the proximal flared portion 44. The struts 49a defining the flare are oriented substantially in the longitudinal direction, to further reduce their impact on attempts to re-cross the ostium 90 with a guidewire.

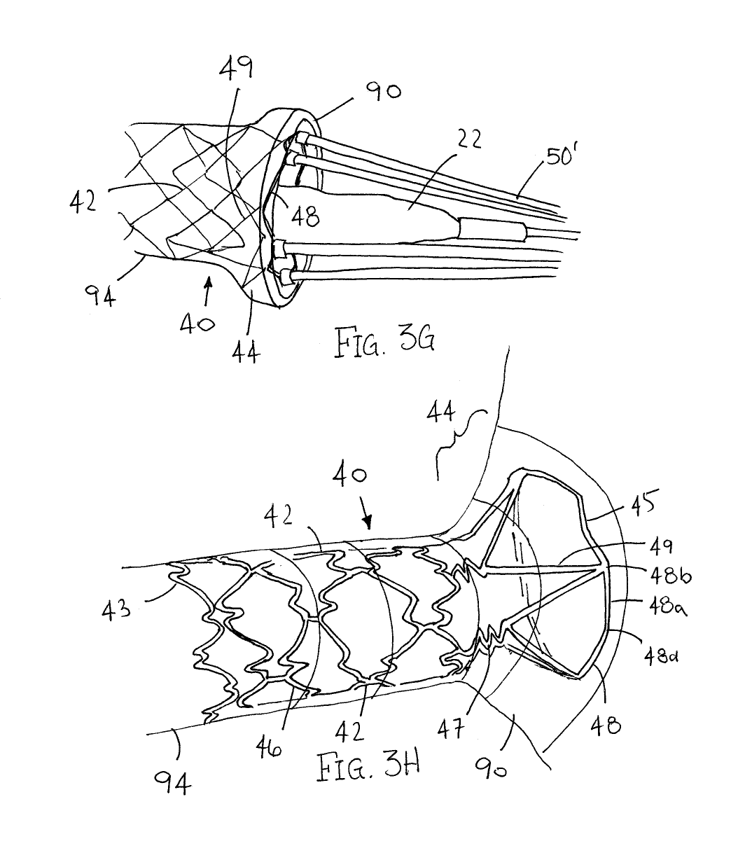

Turning to FIG. 5, another embodiment of an actuator 150 is shown that may be used to flare and/or otherwise deploy the proximal portion 44 of stent 40 (which may be any of the embodiments described herein). Generally, the actuator 150 includes a plurality of arms 154 (one shown) including a slot 155, and a fiber 156. Similar to the embodiment described with reference to FIG. 2, the proximal portion 44 of the stent 40 includes a first row of struts 48, which are attached to a second row of struts 49 via a flexible connector 48c. The struts 48a in the first row 48 are connected at their proximal end to another portion of the stent 40 having one or more flexible connectors 48d, similar to curved elements described above (although shown here with a more complicated geometry). Unlike the previous embodiment, an eyelet 49e is provided adjacent each curved loop 49d in the second row of struts 49.

The curved element 48d of the first row of struts 48 may be received in the slot 155 in the arm 154. The fiber 156, which may be composed of metal, plastic, or other suitable material, is threaded through a hollow bore or other passage of the arm 154, over the curved element 48 positioned in the slot 155, through the eyelet 49e, and back into the hollow bore of the arm 154.

This embodiment of the actuator 150 may allow the proximal portion 44 of the stent 40 to be compressed axially (in the longitudinal direction) by applying a compressive force to the arm 154, while simultaneously applying a tensile force to the fiber 156. In response to the applied stresses, the first and second rows 48, 49 of the proximal portion 44 of the stent 40 may buckle radially outwardly, i.e., in the transverse direction, by bending the flexible connector 48c.

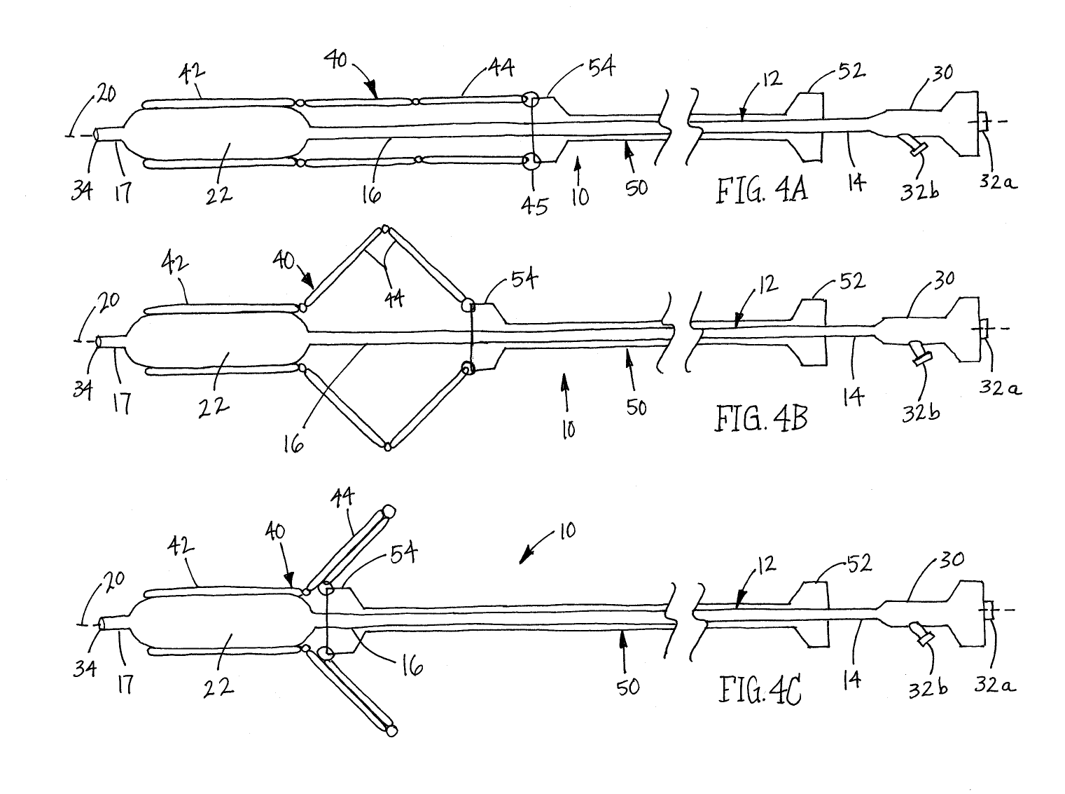

Turning to FIGS. 6A and 6B, another embodiment of an apparatus 210 is shown that includes a delivery catheter 212 and a pusher or actuator 250, which may be similar to the embodiments described elsewhere herein. FIG. 6A shows the apparatus 210 in a condition suitable for tracking through a patient's body to a location in a trunk or other parent vessel. In this embodiment, a proximal balloon 222b is located under a portion of the stent 40 interfacing with the distal end 254 of the pusher 250. The balloon 222b may act as a catch mechanism, e.g., engaging the proximal end 45 of the stent 40, e.g., based upon frictional contact between the balloon 222b and the stent 40, using a low tack adhesive, and the like. Alternatively, the balloon 222b may be inflated or otherwise expanded to provide a stop before deploying the stent 40.

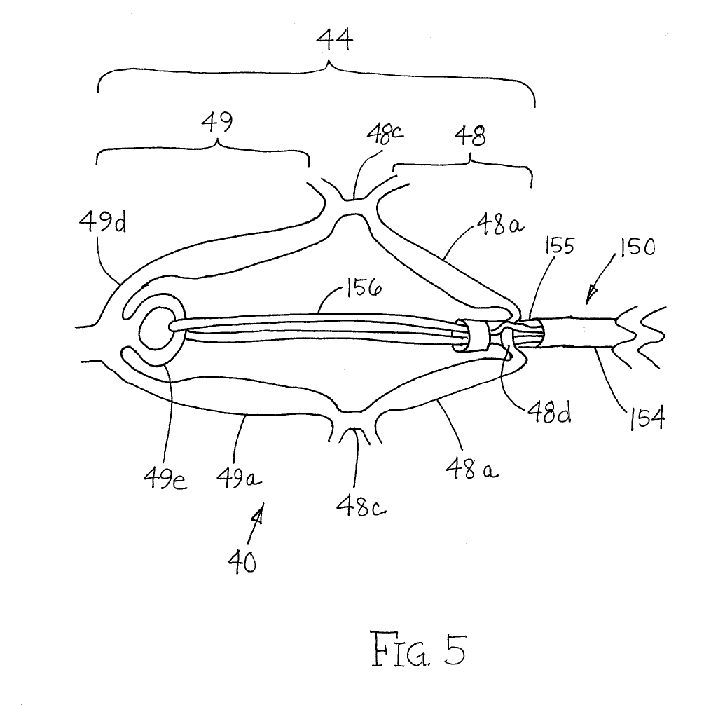

Turning to FIG. 6B, the proximal portion 44 of the stent 40 has been buckled and flared radially outwardly. This may be achieved by advancing pusher 250 distally relative to the stent 40 and distal end 216 of the delivery catheter 212, similar to the previous embodiments described herein. In an alternate embodiment shown in FIG. 7, a portion of the stent 40 is located on the proximal balloon 222b,′ but a reinforcement 256 has been added to the proximal portion of the proximal balloon 222b.′ This reinforcement 256 may act to add additional mechanical integrity to the balloon 222b′ during the linear actuation of the flared portion 44 of the stent 40, e.g., to allow the balloon 222b′ to provide a stop without having to inflate the balloon 222b.′ The reinforcement 256 may simply be a thicker portion of the proximal balloon 222b′ itself, an object embedded inside a wall of the proximal balloon 222b,′ or an object placed inside or adjacent to the proximal balloon 222b,′ e.g., attached to the distal end 216 of the delivery catheter 210. Thus, the balloon 222b′ may provide a stop that compresses the proximal portion 44 of the stent 40, similar to other embodiments described elsewhere herein.

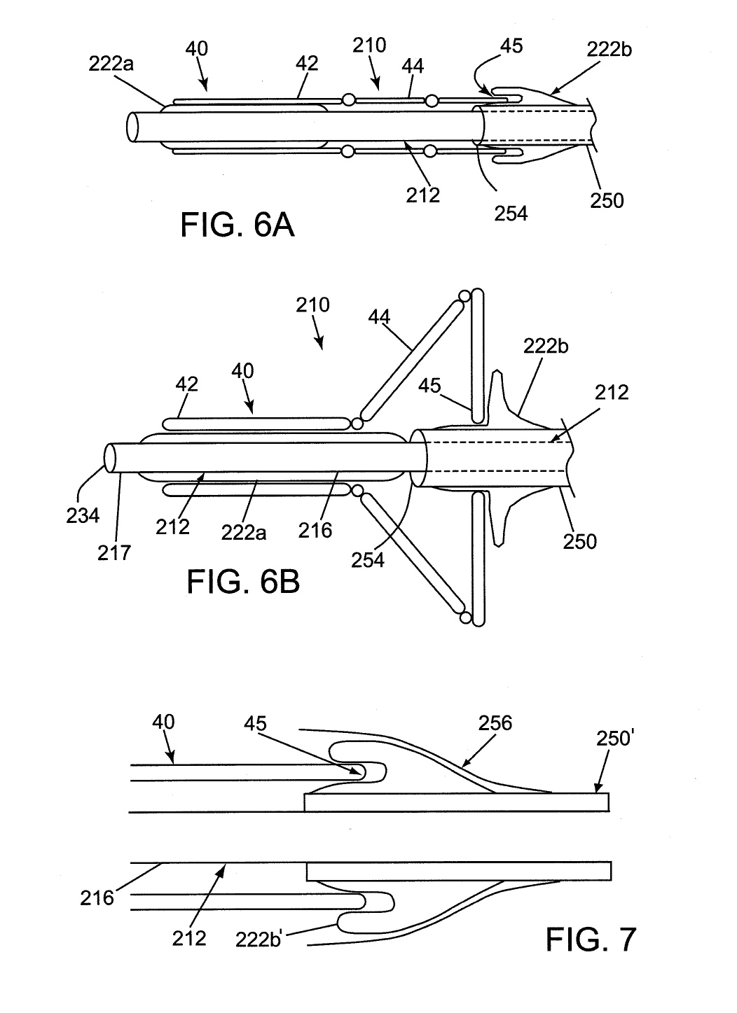

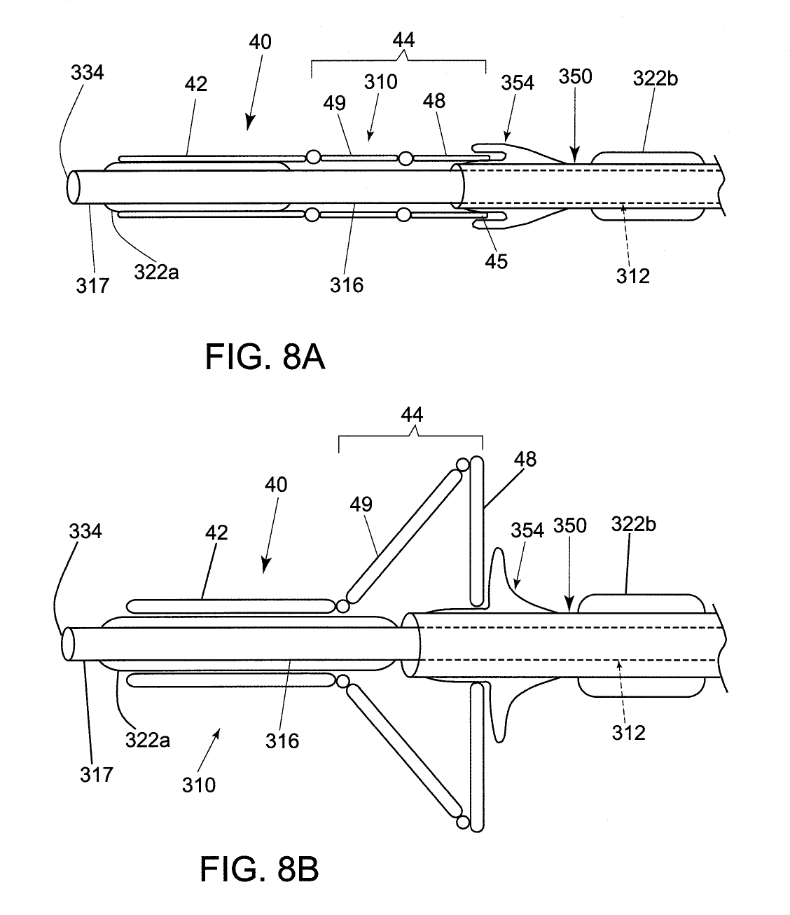

Turning to FIGS. 8A and 8B, a schematic of yet another embodiment of an apparatus 310 is shown including a delivery catheter 312 and a pusher or other actuator 350. The stent 40 shown may be similar to other embodiments described herein, including a distal portion 42 and a proximal portion 44 that includes first and second bands of cells 48, 49 (connected by links or other connectors represented by dots). In this alternative embodiment, the pusher 350 includes a catch mechanism 354 and a proximal balloon 322b attached to and inflatable via the pusher 350. As shown in FIG. 8A, the catch mechanism 354 engages or otherwise contacts a proximal end 45 of the stent 40, e.g., capturing the proximal end 45 between the catch mechanism 354 and a wall of the delivery catheter 312. The stent 40 and balloons 322 are collapsed, allowing the distal end 316 of the delivery catheter 312 to be delivered into a main body lumen (not shown), similar to other embodiments described herein.

As shown in FIG. 8B, the flare on the proximal portion 44 of the stent 40 has been actuated by advancing the pusher 350 until a flared shape is achieved in the proximal portion of the stent (6). The stent 40 is maintained from slipping off of the telescoped tube by the catch mechanism 354, which may resiliently or plastically bend outwardly, as shown, to accommodate flaring of the stent 40. As explained elsewhere herein, the flare of the proximal portion 44 may be actuated in preparation for inserting the stent 40 into an ostium.

Turning to FIG. 8C, the distal balloon 322a may be inflated to expand the distal portion 42 of the stent 40 after proper location in the ostium is achieved using the flared proximal portion 44 of the stent 40.

Next, as shown in FIG. 8D, the distal balloon 322a has been deflated and the pusher 350 has been advanced so that the proximal balloon 322b is disposed under the first row of struts 48 of the proximal portion 44 of the stent 40. The pusher 350 and/or delivery catheter 312 may include tracks, guides, and the like (not shown), which may limit distal movement of the pusher 350, e.g., to place the balloon 322b under the proximal end 45 of the stent 40. When the pusher 350 is advanced, the first row of struts 48 of the proximal portion 44 of the stent 40 may be bent past an angle of ninety degrees (90°) relative to the longitudinal axis 320, which may release the distal end 45 of the stent 45 from the catch mechanism 354. For example, as the first row of struts 48 is bent past ninety degrees (90°), they are no longer under a compressive load, but are under a tensile load.

Turning to FIG. 8E, the proximal balloon 322b may be inflated, causing the proximal portion 44 of the stent 40 to obtain a final flared condition in the ostium, with the first row of struts 48 extending radially, similar to the embodiments described above. Optionally, the distal balloon 322a may remain inflated and/or may be inflated in conjunction with the proximal balloon 322b in order to achieve a desired fully deployed configuration for the stent 40.

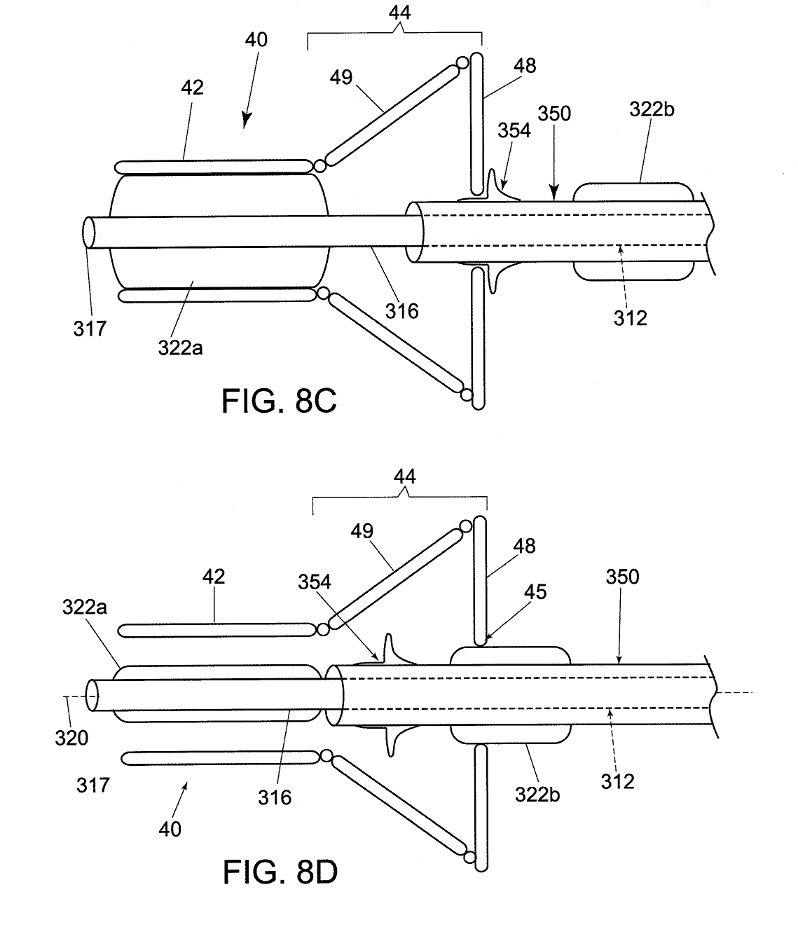

Turning to FIGS. 9A and 9B, still another embodiment of an apparatus 410 is shown that includes a delivery catheter 412 and an actuator 450, which may be constructed generally similar to other embodiments described herein. As shown in FIG. 9A, the delivery catheter 412 may include relatively small fingers, tabs, or catches 458, e.g., formed from metal or other strong material, attached to the distal end 416. The fingers 458 may protrude through the stent 40 to form a mechanical attachment of the stent 40 to the distal end 416 of the delivery catheter 412. These fingers 458 may prevent axial movement of the stent 40 relative to the delivery catheter 412 in the condition shown in FIG. 9A, while allowing the stent 40 to be expanded radially outwardly. Once the balloon(s) 422 are inflated, e.g., as shown in FIG. 9B, the fingers 458 may disengage from the stent 40, releasing the stent 40 from the delivery catheter 412 to allow implantation in the patient.

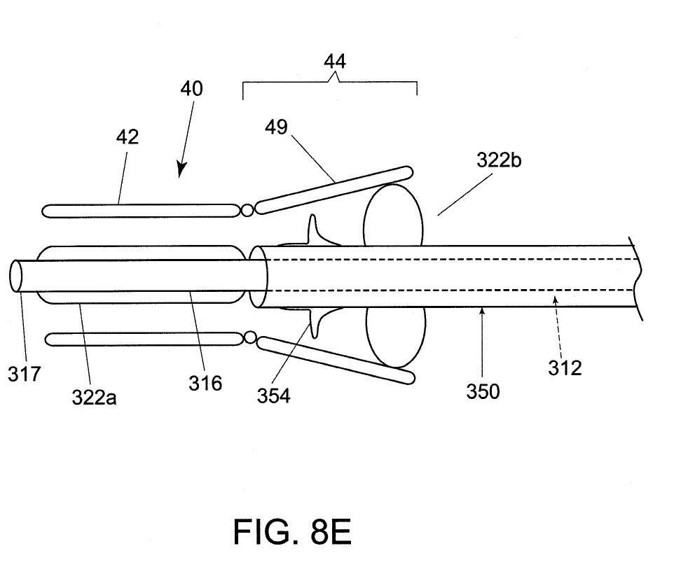

Turning to FIG. 10, a flat-pattern is shown that may be used to cut the fingers 458 from a tube. The diameter of the tube may be chosen to be slightly larger than the distal end 416 of the delivery catheter 412 to aid in crimping, bonding, welding, or otherwise attaching the fingers 458 to the delivery catheter 412. The fingers 458 may be bent radially outwardly from the tube surface to engage features, e.g., cells or struts, of the stent 40 and act as an attachment mechanism. As shown, the tube includes a plurality of additional slits 459. The slits 459 may be useful to allow the tube to be crimped, expanded, or otherwise received over any features that exist on the distal end 416 of the delivery catheter 412, thereby securing the fingers 458 on the distal end 416. The slits 459 are not necessary for the use of the fingers 458.

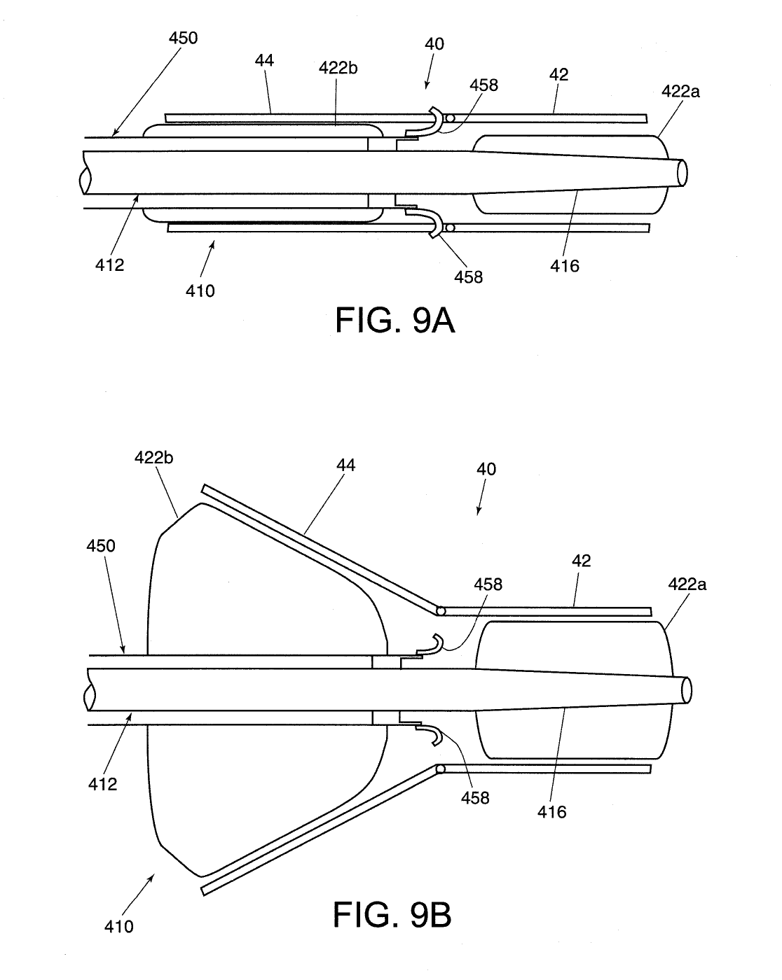

Turning to FIGS. 11A and 11B, the interaction of the fingers 458 with the geometry of a stent 40 is shown. The stent may have a strut 447 oriented in the stent's longitudinal axis that bifurcates into an arc 448. The strut 447 may be inserted between two fingers 458, effectively capturing the stent 40 and preventing axial movement in “Direction 1” shown in FIG. 11A. Upon inflation of the balloon(s) on the delivery catheter 412, the fingers 458 may bend out of the way, allowing the stent 40 to expand radially outward, and become free from the delivery catheter 412. In FIG. 11B, in an alternative embodiment, the finger 458 placed into an eyelet 446 formed into the stent 40. Again, the finger 458 may prevent axial migration of the stent 40, but bend out of the way and release the stent 40 upon balloon inflation.

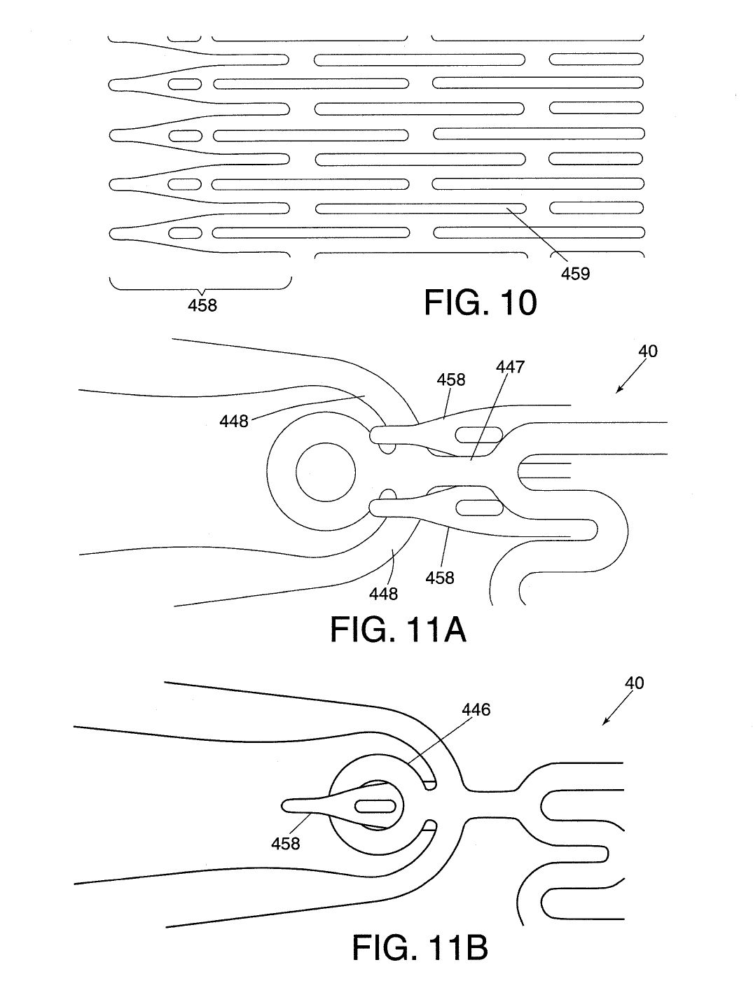

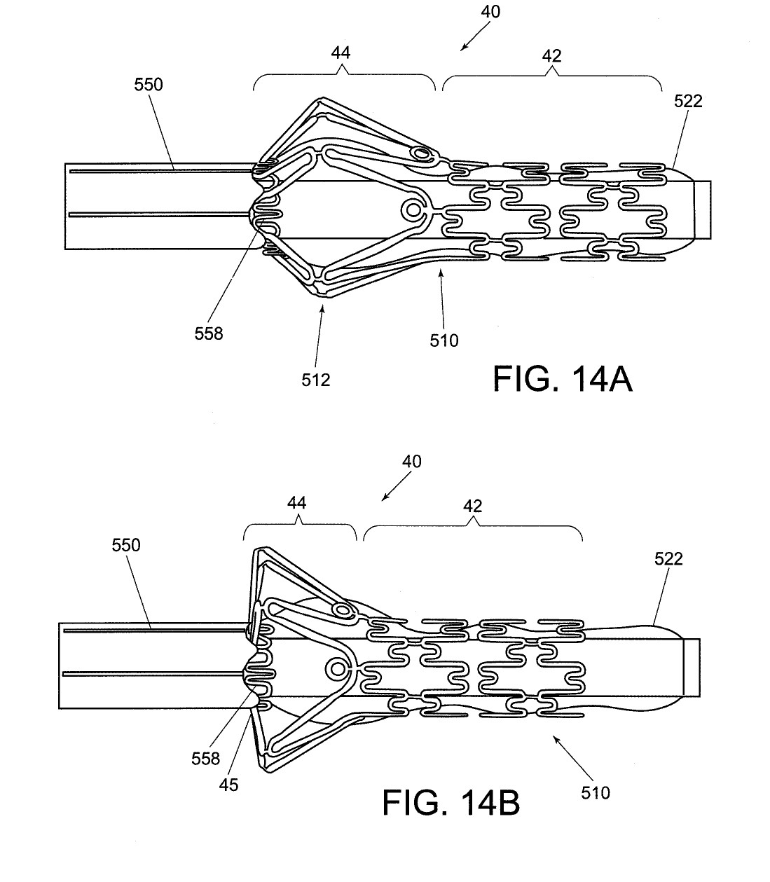

Turning to FIGS. 12-14D, another embodiment of an apparatus 510 is shown that includes a delivery catheter 512, a pusher or actuator 550, and a stent 40, which may be constructed similar to any of the embodiments described elsewhere herein. As shown in FIGS. 14A-14D, the delivery catheter 512 may include a distal end 516 including a balloon 522 thereon and carrying the stent 40. The pusher 550 may include an elongate tubular member and the like (not shown) extending from a proximal end (not shown) of the delivery catheter 512 to the distal end 516, e.g., terminating adjacent the proximal end 45 of the stent 40.

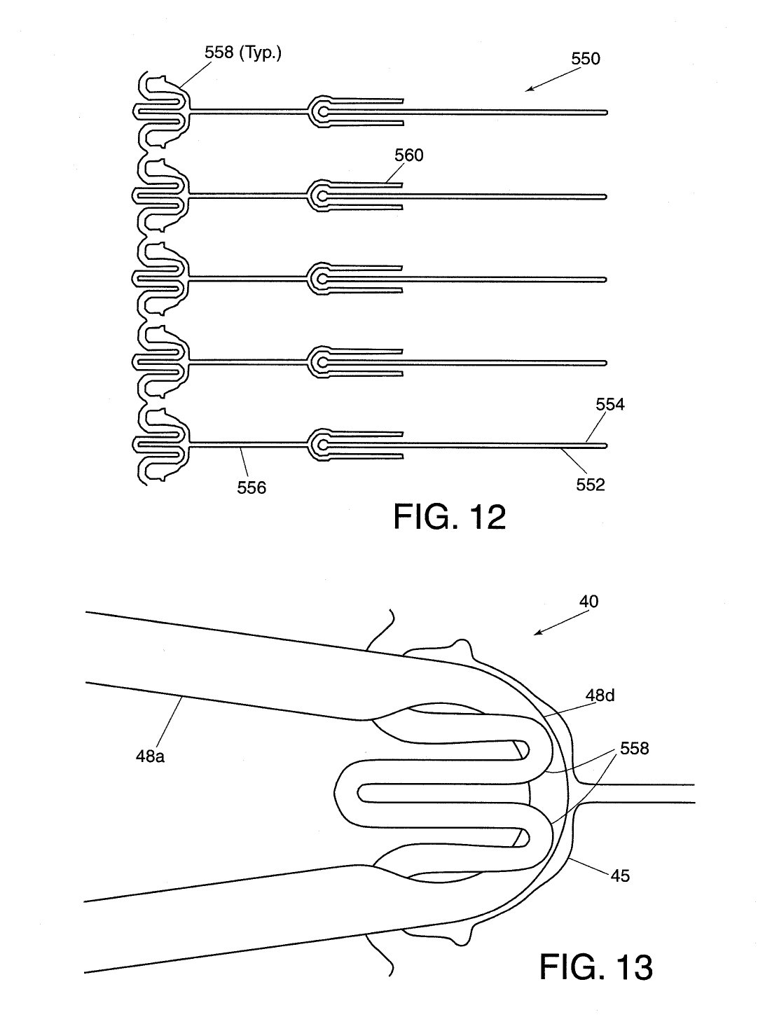

With particular reference to FIGS. 12 and 13, the pusher 550 may include a plurality of connectors 558 on its distal end that may be interlocked or otherwise removably connected to the proximal end 45 of the stent 40. FIG. 12 shows a flat pattern that may be used to cut the distal end of the pusher 550 from a hollow tube of material, e.g., by laser cutting, die cutting, machining, chemical etching, and the like. As shown, the pattern includes a plurality of longitudinal fingers 552 including proximal end 554, which may be connected to the proximal portion (not shown) of the pusher 550, and a distal end 556. The distal ends 556 of the fingers 552 include the connectors 558, which may be cut in a serpentine pattern that is loose relative to the fingers 552. Optionally, the fingers 552 may also contain second stabilizing connectors 560.

Turning to FIG. 13, during use, the stent 40 may be loaded onto the distal end 516 of the delivery catheter 512 (not shown, see FIGS. 14A-14D), and captured using the connectors 558. For example, as shown, curved elements 48d on the proximal end 45 of the stent 40 may be captured under the connectors 558, while axial elements 48a may pass over the connectors 558, thereby providing an interference fit. Thus, the proximal portion 44 of the stent 40 may be limited in axial movement, similar to the previous embodiments.

Turning to FIGS. 14A-14D, deployment of the stent 40 is shown, e.g., after delivering the stent 40 into a trunk adjacent to an ostium, similar to the previous embodiments. As shown in FIG. 14A, the pusher 550 may be advanced to buckle the proximal portion 44 of the stent 40, with the connectors 558 maintaining control of the proximal end 45 of the stent 40 during the linear-actuation used to flare the stent. Turning to FIG. 14B, the proximal portion 44 of the stent 40 has been fully flared condition due to linear actuation, and the connector 558 of the pusher 550 still has control of the proximal end 45 of the stent 40. At this point, if desired, the pusher 550 could be pushed proximally, causing the stent 40 to retract back down to its shape before linear actuation. This may be useful, because, if necessary or desired, the stent 40 may be removed without substantial risk of harming the patient.

Turning to FIGS. 14C and 14D, a proximal balloon 522 on the delivery catheter 512 is shown being inflated, causing the fingers 552 of the pusher 550 to flare out from each other. This, in turn, pulls the connectors 558 out straight from its original serpentine configuration, thereby releasing the proximal end 45 of the stent 40 from the connectors 558. Further, because the connectors 558 allow only limited expansion of the fingers 552, e.g., defined by the length of the serpentine configuration as it straightens, this separates the stent 40 and the pusher 550 as the balloon 522 inflates between them. The flared pusher 550 may also act to mechanically stabilize the balloon 522 in the proximal direction, e.g., when the apparatus 510 is being advanced into an ostium (not shown) and/or during proximal balloon inflation of the stent 40.

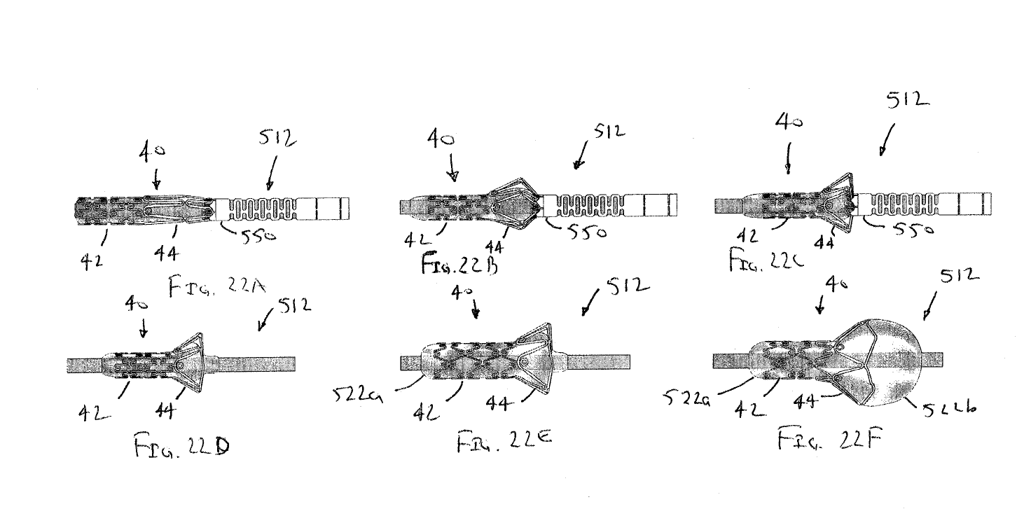

Turning to FIGS. 22A-22F, the various stages of expanding the stent 40 is shown. Although these drawings show the stent 40 being expanded using the delivery catheter 512 and pusher of FIGS. 14A-14D, it will be appreciated that other embodiments described herein may expand the stent 40 using a similar sequence. Initially, in FIG. 22A, the stent 40 is shown in a contracted condition, e.g., for delivery through a patient's vasculature. Similar to the previous embodiments, the stent 40 generally includes a first flaring portion 44 and a second main portion 42. In FIG. 22B, the pusher 550 is being directed distally relative to the delivery catheter 512 and/or the main portion 42, thereby compressing the flaring portion 44 axially. As shown in FIG. 22C, this causes the flaring portion 44 to buckle radially outwardly to an intermediate condition. Optionally, the pusher 550 may be removed, as shown in FIG. 22D (or the pusher 550 is simply omitted for clarity).

Turning to FIG. 22E, a first balloon 522a on the delivery catheter 512 (underlying the main portion 42) may be expanded, thereby causing the main portion 42 to expand from the contracted condition to an enlarged condition. Then, as shown in FIG. 22F, a second balloon 522a on the delivery catheter 512 may be expanded, thereby causing the flaring portion 44 to expand from the intermediate condition to an enlarged condition. Alternatively, the sequence of the expansion of the balloons 522 may be reversed. Alternatively, a single balloon may be provided, and the expansion of the main portion 42 and the flaring portion to the enlarged condition may occur substantially simultaneously.

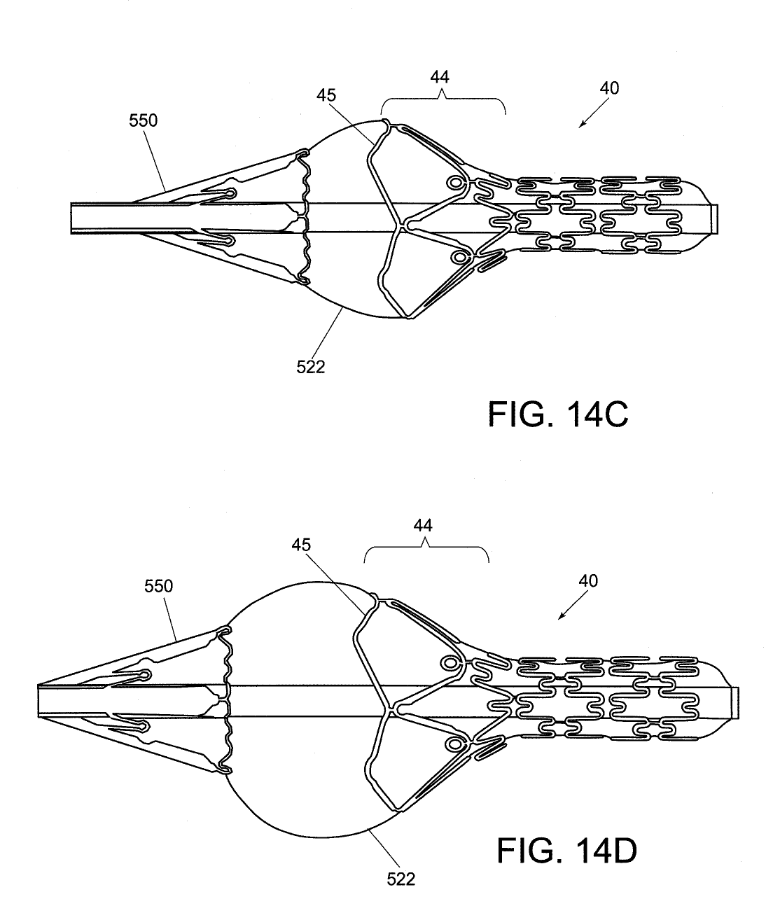

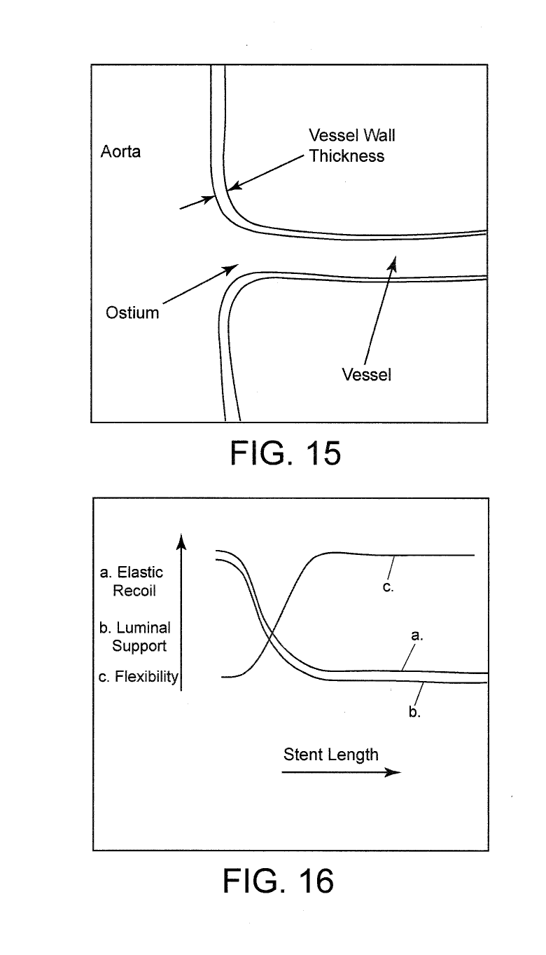

Turning to FIGS. 15 and 16, in any of the embodiments described herein, it may be desirable to have variable properties along a length of the stent, e.g., to accommodate different needs for different portions of a diseased vessel.

For example, FIG. 15 shows a cross-section of a patient's body, including a main vessel, e.g., an aorta, and an ostium communicating with a branch vessel extending from the aorta. As described elsewhere herein, an aorto-ostial lesion may exist within the ostium and/or branch. As can be seen, a thickness of the wall of the vessels may vary from the portion defining the aorta to the portion defining the vessel. This variation in wall thickness provides a situation where a constant design along the stent length is at a disadvantage.

Turning to FIG. 16, expected mechanical properties of an aorto-ostial lesion and the desired mechanical properties of a stent used to treat such an aorto-ostial lesion are shown. The elastic recoil of the vessel (line “a”) starts at a high value due to the thick wall thickness of the vessel near the ostium, and decreases with distance distally into the vessel. To accommodate this high elastic recoil, the desired stent luminal support (line “b”) may mimic the elastic recoil of the vessel. If a constant luminal support stent design were deployed, the designer would have to choose a luminal support that was either too weak to address the high elastic recoil of the vessel near the ostium, or too strong (and potentially damaging) to the distal portion of the vessel.

In addition, flexibility is also shown (line “c”) in FIG. 16. In general, flexibility is always desired in a stent, but flexibility often comes by reducing the luminal support of a stent. For this reason, the desired flexibility is shown as an inverse function of the luminal support, having low flexibility near the ostium, and greater flexibility in the distal portion of the vessel.

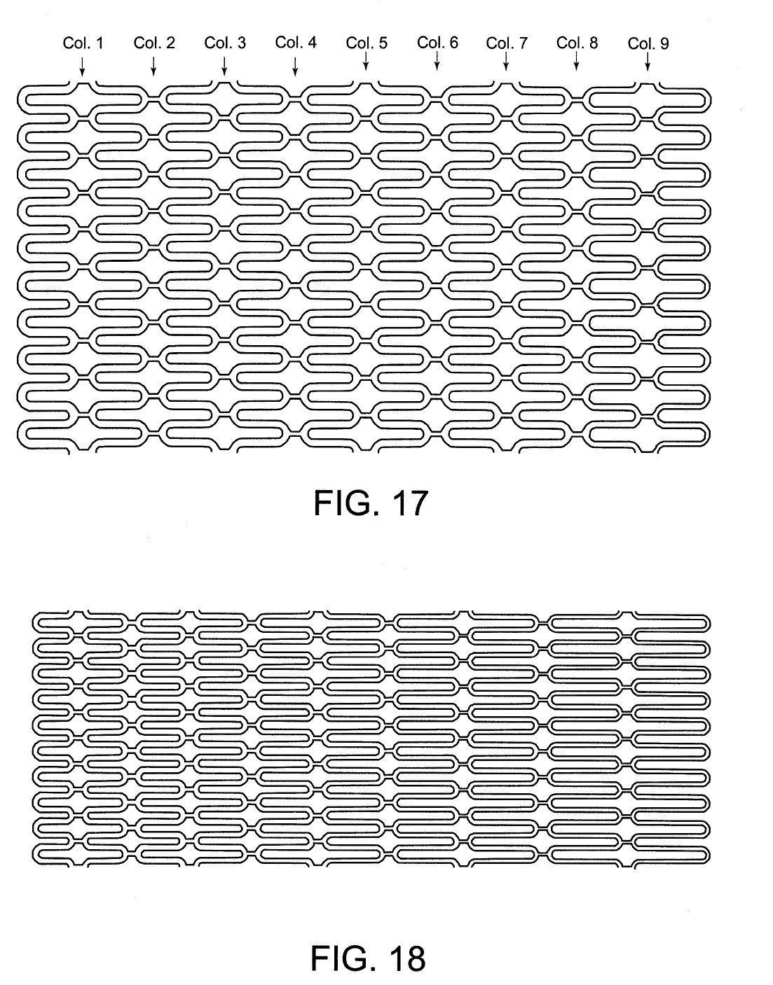

Turning to FIG. 17, an exemplary embodiment of a cell pattern is shown that may be used to provide variable luminal support and flexibility, e.g., for the reasons just discussed. The exemplary cell pattern shown includes nine (9) columns of cells. The cells are defined as having a serpentine pattern along each of the cells' two sides, and a connector defining the top and bottom of each cell. The thicknesses of the serpentine patterns and connectors have been made such that Column 1 has the highest thickness and Column 9 has the lowest thickness. By varying the thickness of the straight portions of the serpentine pattern, the bent portions of the serpentine pattern, and the connectors, the stent may be made to have a greater luminal support in the columns of cells having thicker cell elements than those rows of cells having thinner cell elements.

Alternatively, as shown in FIG. 18, a cell pattern may be provided for a stent that includes variable cell width. Because the serpentine patterns provide the majority of luminal support, and the serpentine patterns are closer together in cells with smaller widths than larger widths, a gradient of luminal support may be achieved. In the case of the ostium shown, the area of the stent adjacent to the ostium would have small cell widths, and the cells would become wider distally along the length of the stent.

Turning to FIG. 19, yet another cell pattern is shown where the number of connectors between cells has been varied along the length of the stent. By varying the number of connectors in each column of cells, the flexibility of the stent may be modified. For example, those rows with less connectors may be more flexible than those columns having more connectors. In addition, it is envisioned that luminal support may be higher in rows with more connectors because it is more constrained in how it may bend under elastic recoil loads. The number of connectors, therefore, may also enable varying the mechanical properties of the stent as needed for specific lesion types.

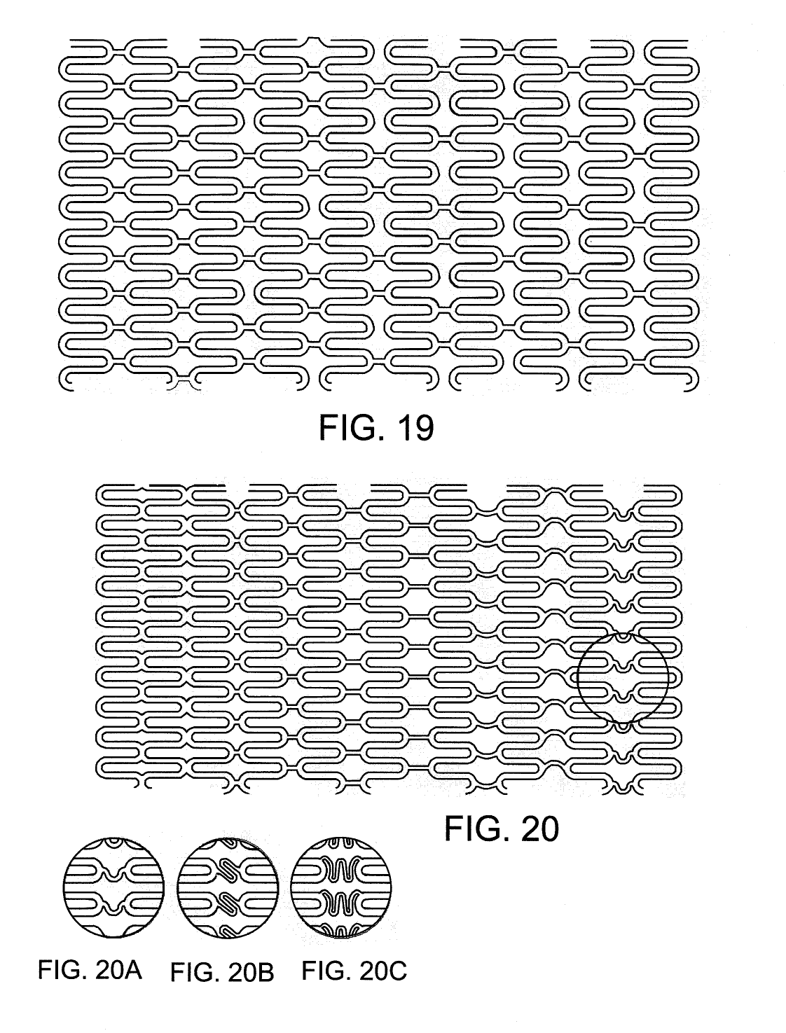

In another embodiment, shown in FIG. 20, a cell pattern may be provided where the connector design varies from one side of the stent to the other. As shown, the left column has the bent portion of the serpentine pattern merged into the adjacent serpentine pattern. This may create a cell structure that has a high degree of luminal support and low flexibility due to the high degree of deformation that must occur in the small area contained in the junction between serpentine patterns. The adjacent cells show a gradual dissociation of serpentine patterns and the creation of a connector that bridges the bent portion of adjacent serpentine patterns. These cells may decrease in luminal support and increase in flexibility as the connector becomes more defined and longer.

The right-most cells show the creation and exaggeration of a bend in the connector. As the connector becomes bent to a greater degree from the longitudinal axis of the stent, it may become easier to bend under compressive, axial loads, and also may become capable of elongating in the axial direction under tensile, axial loads. In general, bending the flexible connector to a greater degree may make it more compliant. This increase in connector compliance may reduce the luminal support of the stent, and increase its flexibility. In addition to a single bend in the connector as shown in FIG. 20A, additional bends can be designed into the connectors as shown in FIGS. 20B and 20C.

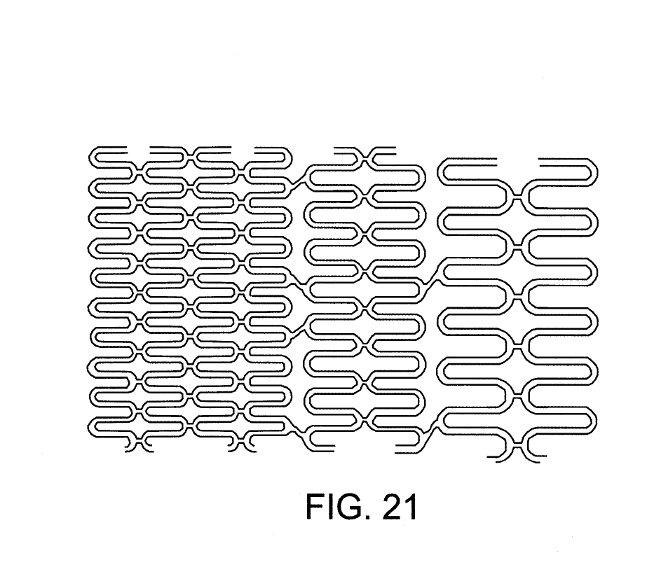

Finally, as shown in FIG. 21, it may be possible to vary the mechanical properties of a stent along its length by varying the number of serpentine convolutions around its circumference and/or varying the diameter of the radii of the bent portion of the serpentine convolutions. As shown, the first and second leftmost columns each have ten (10) cycles in their serpentine convolutions, while third column has eight (8) and the fourth column has six (6). This reduction in the number of serpentine convolutions alone, or in conjunction with the other methods described above may be used to vary the mechanical properties of the stent along its length.

In addition, the radius of the serpentine convolutions may be varied to change the mechanical properties of the stent along its length. For example, as shown, the first and second leftmost columns have a radius of that is smaller than the third column, which has a radius smaller than the fourth column. Generally, larger radii may allow more uniform stress distribution, and lower forces to deform the stent. This property, however, may also be combined with the other designs for varying the mechanical properties of a stent along its length. Thus, it will be appreciated that any of these combinations may be utilized alone or together to provide a stent having desired mechanical properties along its length, such as those shown in FIG. 15.

It will be appreciated that elements or components shown with any embodiment herein are exemplary for the specific embodiment and may be used on or in combination with other embodiments disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

We claim:

1. A stent, comprising:
a first tubular portion comprising a length, the first tubular portion being expandable from a contracted condition to an expanded condition;
a second tubular portion comprising a first annular band disposed adjacent the first tubular portion and a second annular band disposed adjacent the first tubular portion, the second tubular portion being configured such that upon application of an axial compressive force, the first and second annular bands buckle outwardly at a location between the first and second annular bands, the second tubular portion further configured such that the second annular band expands into a ring upon application of a radially outward expansion force.

2. A stent, comprising:
a first tubular portion comprising a length, the first tubular portion being expandable from a contracted condition to an expanded condition;
a second tubular portion comprising a first annular band disposed adjacent the first tubular portion and a second annular band disposed adjacent the first tubular portion, the second annular band comprising a plurality of axial elements connected by alternating curved elements, the second tubular portion being configured such that upon application of an axial compressive force, the first and second annular bands buckle outwardly at a location between the first and second annular bands, the second annular band configured such that, upon application of a radially outward expansion force, the curved elements at least partially straighten such that the axial elements at least partially define a circle or ellipse.

3. An apparatus for treating an ostium communicating between a main body lumen and a branch body lumen, comprising:
an elongate member comprising a proximal end, a distal end sized for introduction into the main body lumen and the branch body lumen;
a stent on the distal end that is expandable between contracted and enlarged conditions, the stent comprising a first flaring portion, and a second main portion; and
an actuator movable relative to the distal end for buckling the first flaring portion of the stent when the actuator is activated, the first flaring portion comprising first and second annular bands, the first flaring portion configured to buckle radially outwardly between the first and second annular bands when the actuator is activated, thereby defining an intermediate condition.

4. The apparatus of claim 3, further comprising a first expandable member, the second main portion of the stent overlying the first expandable member such that the second main portion is expanded from the contracted to the enlarged condition when the first expandable member is expanded.

5. The apparatus of claim 4, further comprising a second expandable member on the distal end adjacent the first expandable member, the second expandable member being expandable from a collapsed configuration to an expanded configuration for expanding the first flaring portion radially outwardly from the intermediate condition to the enlarged condition.

6. The apparatus of claim 5, wherein the first flaring portion comprises a plurality of struts that extend substantially axially in the contracted condition, the plurality of struts extending outwardly in the intermediate condition and extending circumferentially in the enlarged condition.

7. The apparatus of claim 3, wherein the actuator comprises a pusher member movable axially between proximal and distal positions, the pusher member being disposed adjacent the first flaring portion in the proximal position and pushing against the first flaring portion as the pusher member is moved towards the distal position, thereby causing the first flaring portion to buckle radially outwardly.

8. The apparatus of claim 3, wherein the actuator comprises a plurality of arms that contact a proximal end of the stent while the first flaring portion of the stent is buckled when the actuator is activated.

9. The apparatus of claim 8, wherein the plurality of arms comprise features that releasably engage the proximal end of the stent.

10. The apparatus of claim 9, wherein the actuator is deactivatable for returning the first flaring portion from the intermediate condition towards the original contracted condition.

11. The apparatus of claim 9, wherein the features comprise fingers that interlock with the proximal end of the stent.

12. The apparatus of claim 3, further comprising one or more features on the distal end of the elongate member for substantially securing the second main portion when the actuator is activated.

13. A method for delivering a stent within an ostium communicating between a main body lumen and a branch body lumen, the method comprising:
introducing a stent into the main body lumen with the stent in a contracted condition, the stent comprising a first flaring portion, and a second main portion;
compressing the stent, thereby causing the first flaring portion to buckle radially outwardly to an intermediate condition;
advancing the stent into the ostium with the first flaring portion in the intermediate condition;
expanding the second main portion within the branch body lumen to an enlarged condition; and
expanding the first flaring portion from the intermediate condition to an enlarged condition.

14. The method of claim 13, wherein the second main portion is expanded to the enlarged condition before the first flaring portion is expanded to the enlarged condition.

15. The method of claim 13, wherein the first flaring portion is expanded to the enlarged condition substantially simultaneously when the second main portion is expanded to the enlarged condition.

16. The method of claim 13, wherein the stent is compressed using an actuator.

17. The method of claim 16, wherein the actuator comprises a plurality of arms that releasably engage a proximal end of the stent, the plurality of arms being movable towards the second main portion of the stent when the actuator is used to compress the stent.

18. The method of claim 13, wherein the stent is released from the plurality of arms when at least one of the first flaring portion and the second main portion is expanded to the enlarged condition.

19. A method for delivering a stent within an ostium communicating between a main body lumen and a branch body lumen, the method comprising:
providing a stent on a distal end of a delivery device, the stent comprising a first flaring portion and a second main portion;
introducing the distal end and the stent into the main body lumen with the stent in a contracted condition; and
activating an actuator on the delivery device, thereby compressing the first flaring portion of the stent, thereby causing the first flaring portion to buckle radially outwardly to an intermediate condition.

20. The method of claim 19, further comprising deactivating the actuator, thereby causing the first flaring portion to compress from the intermediate condition back towards the contracted condition.

21. The method of claim 20, further comprising removing the distal end and the stent from the main body lumen.
